(19) United States
(12) Patent Application Publication
(10) Pub. No.: US 2004/0147393 A1
(43) Pub. Date: Jul. 29, 2004

(54) Preparation of a multimetal oxide composition

(75) Inventors: Hartmut Hibst, Schriesheim (DE); Frieder Borgmeier, Mannheim (DE); Klaus Joachim Muller-Engel, Stutensee (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Akiengesellschaft, Ludwigshafen (DE)

(21) Appl. No.: 10/647,330

(22) Filed: Aug. 26, 2003

Related U.S. Application Data
(60) Provisional application No. 60/476,165, filed on Jun. 6, 2003.

(30) Foreign Application Priority Data
Jan. 29, 2003 (DE) 10303526.5

(51) Int. Cl. C07C 61/10; B01J 23/00

(52) U.S. Cl. 502/302; 502/313; 502/314

(57) ABSTRACT

A process for preparing a multimetal oxide composition comprising one of the elements Mo and V and at least one of the elements Te and Sb, in which part solutions which each contain partial amounts of the required starting compounds of the elemental constituents present in the multimetal oxide composition in dissolved form are prepared from the starting compounds, these part solution streams are combined and mixed and the resulting mix solution stream is broken up into fine droplets, dried by means of a hot gas and the solid obtained is treated thermally at elevated temperature.

The present invention relates to a process for preparing a multimetal oxide composition M of the stoichiometry I

Mo₁VₐM¹_{b}M²_{c}M³_{d}M⁴ₑOₙ (I),

M¹=at least one element from the group consisting of Te and Sb;

M²=at least one element from the group consisting of Nb, Ti, W, Ta, Bi, Zr and Re;

M³=at least one element from the group consisting of Pb, Ni, Co, Fe, Pd, Ag, Pt, Cu, Au, Ga, Zn, Sn, In, Ce, Ir, Sm, Sc, Y, Pr, Nd and Tb;

M⁴=at least one element from the group consisting of Li, Na, K, Rb, Cs, Ca, Sr, Ba;

a=0.01 to 1,

b=≧0 to 1,

c=>0 to 1,

d=≧0 to 0.5,

e=≧0 to 1 and

n=a number which is determined by the valence and abundance of elements other than oxygen in (I),

in which a mix solution is produced continuously in a solvent from the required starting compounds of the elemental constituents of the multimetal oxide composition M, the mix solution is fed continuously into a drying apparatus for removing the solvent and the solid obtained is treated thermally at elevated temperature, with the thermal treatment comprising a calcination at from 200 to 1 200° C.

Multimetal oxide compositions of the stoichiometry I and processes for preparing them are known (cf., for example, EP-A 318295, EP-A 512846, EP-A 767164, EP-A 895809, EP-A 529853, EP-A 608838, EP-A 962253, DE-A 10248584, DE-A 10119933 and DE-A 10118814). They are suitable as catalytically active compositions for heterogeneously catalyzed partial gas-phase oxidation and/or ammoxidations of saturated and unsaturated hydrocarbons and of lower aldehydes, as described, for example, in the abovementioned documents.

If propane and/or propene are/is used as hydrocarbon, it is possible to produce, for example, acrolein, acrylic acid and/or acrylonitrile as target compounds. Acrolein can itself be used as starting compound for producing the latter two compounds. These target compounds are important intermediates which are used, for example, for preparing polymers which can be used as, for example, adhesives. Correspondingly, methacrolein and methacrylic acid are obtainable from isobutane and isobutene. Methacrolein can also be a starting compound for preparing methacrylic acid. The preparation of multimetal oxide compositions of the stoichiometry I is usually carried out by producing an intimate dry mix from starting compounds containing their elemental constituents and treating this thermally at elevated temperature. Possible starting compounds or sources of the elemental constituents are essentially all those which are able to form oxides and/or hydroxides on heating (if necessary in air). Of course, oxides and/or hydroxides of the elemental constituents can be used in part or exclusively as such starting compounds.

In EP-A 529853 and EP-A 608838, at least two physically separate part solutions containing the required starting compounds of the elemental constituents of the multimetal oxide composition are firstly prepared, the part solutions are combined with one another, the resulting mixture is dried and the solid obtained on drying is treated thermally, with the process employed for drying being able to be selected freely. Although a comparatively intimate dry mix of the sources can be produced by the route via the part solutions, a disadvantage of the procedure described in EP-A 529853 and EP-A 608838 is that combining the part solutions as described in these documents normally does not result in a mix solution but instead a suspension comprising partial amounts of the constituents as solids.

This is a disadvantage because the solid present in the suspension generally has a composition which is different from that of the material dissolved in the dispersion medium. For this reason, drying of such a suspension normally does not give a chemically homogeneous solid but a solid whose chemical composition displays some variation in physical space, which normally can no longer be completely eliminated during the subsequent thermal treatment and reduces the catalytic activity of the resulting active compositions.

According to EP-A 603836, an improvement can be achieved by employing the spray drying process for drying the suspension, since drying by evaporation will result in the material dissolved in the dispersion medium being additionally precipitated in fractionated form according to the respective solubilities and additionally emphasize the stated inhomogeneity.

According to EP-A 962253 and according to the applicant's own observations, the precipitation which forms the suspension in the described preparation of multimetal oxide compositions of the stoichiometry I occurs with some time delay after the part solutions have been combined. This time delay can be increased by the part solutions being diluted with solvent and/or cooled. In this way, according to EP-A 962253, a sufficiently stable mix solution can be produced at the beginning and this mix solution can be dried in a manner known per se. However, this procedure has the disadvantage that any use of an increased amount of solvent increases the energy consumption during drying. Furthermore, it increases the drying time and thus the risk of fractional precipitation during drying. Cooling of the part solutions likewise increases the energy consumption required for drying and, in addition, gives only a limited advantage since although this measure does slow the kinetics of the precipitation process, it at the same time reduces the solubilities in the great majority of cases, which promotes undesirable precipitation.

Similarly, JP-A 7-315842 recommends prior preparation of a mix solution so that this can be dried so quickly that the solvent is removed before fractional precipitation can occur.

However, in the case of a previously prepared quantity of solution, precipitation generally commences before the total amount of it had been dried, which leads to losses. This applies particularly when industrial-scale batches are involved.

JP-A 7-315842 therefore expresses the desire for a process in which the mix solution is produced continuously and the mix solution produced is fed by means of a pump directly to solvent removal and is dried there before partial precipitation can occur.

However, a disadvantage of JP-A 7-315842 is that it is not able to make such a desired process available.

JP-A 11-306228 even considers such a procedure to be more or less impossible and therefore aims to form the suspension at least continuously and as homogeneously as possible since formation of a suspension is unavoidable.

It is an object of the present invention to provide a process conforming to the desire expressed in JP-A 7-315842.

We have found that this object is achieved by a process for preparing a multimetal oxide composition M of the stoichiometry I

Mo₁VₐM¹_{b}M²_{c}M³_{d}M⁴ₑOₙ (I),

M¹=at least one element from the group consisting of Te and Sb;

M²=at least one element from the group consisting of Nb, Ti, W, Ta, Bi, Zr and Re;

M³=at least one element from the group consisting of Pb, Ni, Co, Fe, Pd, Ag, Pt, Cu, Au, Ga, Zn, Sn, In, Ce, Ir, Sm, Sc, Y, Pr, Nd and Tb;

M⁴=at least one element from the group consisting of Li, Na, K, Rb, Cs, Ca, Sr, Ba;

a=0.01 to 1,

b=≧0 to 1,

c=>0 to 1,

d=≧0 to 0.5,

e=≧0 to 1 (or ≧0 to 0.5) and

n=a number which is determined by the valence and abundance of elements other than oxygen in (I),

in which a mix solution is produced continuously in a solvent from the required starting compounds of the elemental constituents of the multimetal oxide composition M, the mix solution is fed continuously into a drying apparatus for removing the solvent and the solid obtained is treated thermally at elevated temperature, with the thermal treatment comprising a calcination at from 200 to 1 200° C., wherein at least two physically separate part solutions each containing partial amounts of the required starting compounds of the elemental constituents of the multimetal oxide composition M in dissolved form are firstly prepared, at least two part solution streams are produced from the two or more part solutions, the two or more part solution streams are combined to form a total solution stream, the total solution stream is passed through a mixing zone in which a mix solution stream comprising the total amount of the required starting compounds in dissolved form is formed, the mix solution stream is either broken up into fine droplets in the mixing zone or the mix solution stream is discharged from the mixing zone and then broken up into fine droplets, the fine droplets of mix solution are dried by contact with hot gas and the solid obtained is treated thermally at elevated temperature, with the thermal treatment comprising a calcination at from 200 to 1 200° C.

In contrast to the total solution stream, the mix solution stream has an essentially homogeneous composition.

An important difference between the process of the present invention and the procedure considered desirable in JP-A 7-315842 is that according to JP-A 7-315842 it is not a mix solution stream but initially only a mix solution which is produced directly and this then firstly has to be converted into a mix solution stream by means of a pump and transported to drying.

However, it is advantageous according to the present invention to convert the partial amounts of the part solutions in which the required starting compounds are present in dissolved form into part solution streams and, according to the present invention, to transform them into a mix solution stream. The background to this advantageous nature is, inter alia, the fact that it is virtually always possible to prepare stable part solutions from partial amounts of the starting compounds required for preparing the multimetal oxide compositions M, while the mix solution containing the total amount of the starting compounds required for preparing the multimetal oxide composition M is normally thermodynamically unstable even in a highly diluted state and sooner or later forms sparingly soluble mix compounds which precipitate as solids until their solubility limit is reached. Thus, the part solutions can, in the process of the present invention, be prepared discontinuously beforehand in any batch size and subsequently be converted into the appropriate part solution streams or into the mix solution stream. Of course, the part solutions can also be produced continuously in the process of the present invention.

As sources of the elemental constituents of the multimetal oxide composition M in the process of the present invention, it is in principle possible to use all those which are able to form oxides and/or hydroxides on heating (if necessary in air). Of course, oxides and/or hydroxides of the elemental constituents can also be used as part or all of such starting compounds. Apart from oxides and/or hydroxides, possible sources of the elemental constituents in the process of the present invention are, in particular, salts of organic and/or inorganic acids. Examples which may be mentioned are halides such as chlorides, formates, acetates, oxalates, carbonates, nitrates, sulfates and sulfites.

Sources of the element Mo suitable for the purposes of the present invention are, for example, molybdenum oxides such as molybdenum trioxide, molybdates such as ammonium heptamolybdate tetrahydrate and molybdenum halides such as molybdenum chloride. Suitable starting compounds for the element V which can be used according to the present invention are, for example, vanadium oxysulfate hydrate, vanadyl acetylacetonate, vanadates such as ammonium metavanadate, vanadyl oxalate, vanadyl sulfate, vanadium oxides such as vanadium pentoxide (V₂O₅), vanadium halides such as vanadium tetrachloride (VCl₄) and vanadium oxyhalides such as VOCl₃. It is also possible to use vanadium starting compounds in which the vanadium is present in the oxidation state +4.

Suitable sources for the element tellurium include, according to the present invention, tellurium oxides such as tellurium dioxide, metallic tellurium, tellurium halides such as TeCl₂, and also telluric acids such as orthotelluric acid H₆TeO₆.

Sources of niobium which are suitable for the purposes of the present invention are, for example, niobium oxides such as niobium pentoxide (Nb₂O₅), niobium oxyhalides such as NbOCl₃, niobium halides such as NbCl₅, and also complexes of niobium with organic carboxylic acids and/or dicarboxylic acids, e.g. oxalates, citrates, tartrates and alkoxides or their ammonium salts such as niobium ammonium citrate, niobium ammonium oxalate, niobium ammonium tartrate, etc. Of course, the Nb-containing solutions used in EP-A 895809 are also suitable as niobium source.

As regards all other possible elements (in particular Pb, Ni, Cu, Co, Fe, Bi and Pd and the alkali metals and alkaline earth metals), suitable starting compounds include, in particular, their halides, nitrates, formates, oxalates, acetates, carbonates and/or hydroxides. Further suitable starting compounds frequently also include their oxo compounds such as tungstates or the acids derived therefrom. Ammonium salts are frequently also used as starting compounds here.

Further possible starting compounds are polyanions of the Anderson type, as are described, for example, in Polyhedron Vol. 6, No. 2, pp. 213-218, 1987. A further suitable literature source for polyanions of the Anderson type is Kinetics and Catalysis, Vol. 40, No. 3, 1999, pp. 401 to 404.

Other polyanions suitable as starting compounds are, for example, those of the Dawson or Keggin types.

Of course, the part solutions required according to the present invention can also be prepared using complexing agents which volatilize and/or decompose during the thermal treatment of the dried solid (e.g. complexation as oxalate, citrate and/or acetylacetonate).

Furthermore, the acidic or basic character of the part solutions can also be altered in a targeted manner by addition of organic and/or inorganic acids or bases in order to exert a targeted influence on the solubility behavior. As a further parameter, the temperature during the preparation of the part solutions can be increased or reduced, depending on what has an advantageous effect on the solubility (can be determined in a few preliminary experiments).

According to the present invention, the solvent used is preferably an aqueous solvent or water only. However, it is in principle also possible to use alcohols such as methanol and ethanol and also organic and/or inorganic acids, e.g. acetic acid. Mixtures of the abovementioned solvents, in particular aqueous mixtures, can naturally also be used.

The solids content of the mix solution to be prepared according to the present invention can vary. Expressed as total content of the metals present in the respective mix solution, it is normally at least 0.01% by weight, frequently at least 0.1% by weight, usually at least 1% by weight, often at least 5% by weight and if possible at least 10% by weight, in the process of the present invention. In general, this solids content will not exceed 30% by weight. It is frequently not more than 25% by weight or not more than 20% by weight (the percentage by weight is in all cases based on the weight of the respective mix solution (or of the respective mix solution stream)). Correspondingly, like the solids content of the mix solutions, the corresponding solids content of the part solutions can also vary, i.e. the abovementioned figures are also valid for the part solutions. In particular, the abovementioned solids contents apply to aqueous part solutions or mix solutions. While the solids content as defined above of the mix solution will generally not exceed the 30% by weight limit, solids contents of the part solutions (aqueous and nonaqueous) of up to 50% by weight and more are possible. Such high solids contents of a part solution will normally be compensated in the mix solution by means of at least one part solution having a lower solids content being used to make up the mix solution. Of course, different solvents can also be used for the part solutions to be combined according to the present invention (e.g. water for one part solution and methanol or ethanol or alcoholic aqueous mixtures for another part solution).

Correspondingly, the temperature of the part solution streams to be combined to form a total solution stream can be identical or different in the process of the present invention. The temperature of a part solution stream (in particular an aqueous stream) in the process of the present invention will normally be in the range from ≧0° C. to ≦100° C., preferably from ≧5° C. to ≦80° C., particularly preferably from ≧10° C. to ≦60° C., very particularly preferably from ≧15° C. to ≧40° C. and advantageously in terms of use from 20° C. to 30° C.

In the process of the present invention, the part solution streams can be conveyed under atmospheric pressure, under superatmospheric pressure or using reduced pressure.

Quite generally, for the purposes of the present text, the term solution refers to a system which is liquid, optically transparent and (apart from any solid diluents added) free of solids and precipitates.

The number of part solutions streams has to be at least two in the process of the present invention. However, it can be three, four, five or six, seven, eight or nine or ten. The number of part solution streams is advantageously not more than five in the process of the present invention.

The sources of the elemental constituents of the multimetal oxide composition M which form a suitable partial amount of the total starting compounds required for preparing the multimetal oxide composition M to be dissolved in a part solution needs to be decided on a case-by-case basis and can be determined by a person skilled in the art by means of a few preliminary tests.

If the multimetal oxide composition M comprises the element Nb, it is frequently advantageous in the process of the present invention to dissolve this element in a separate part solution. On the other hand, there are normally no difficulties in dissolving the elements Mo, V and Te together in a single part solution.

The two or more part solution streams can be combined as necessary for the purposes of the present invention by, for example, firstly converting the part solutions from at least two reservoirs containing the two or more part solutions by means of an appropriate number of pumps into physically separate, continuously flowing part solution streams which are conveyed in separate lines (in the simplest case hoses or tubes).

In the simplest case, the two or more part solution streams are then conveyed to the two inlets of a T-piece (the feed lines preferably narrow in the inlet part of the T-piece).

In the interior of the T-piece, the two part solution streams combine and flow together as a total solution stream into the outlet part of the T-piece via which the total solution stream is conveyed out of the T-piece

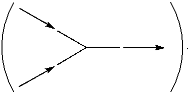

After the two part solution streams have been combined, they are mixed essentially homogeneously while they are being conveyed onwards as a total solution stream. This mixing can, for example, be due predominantly to the turbulence generated when the streams are combined.

A static mixer (e.g. one of the SMXS type from Sulzer Chemtech, D-61239 Ober-Morlen-Ziegenberg) and/or a dynamic mixer, for example, can also be integrated into the outlet part so that the total solution stream flows through this and leaves it as an essentially homogeneous mix solution stream. Static and dynamic mixers are in principle spaces containing static or moving obstacles which influence the flow of the mix solution stream so as to generate turbulence which effects mixing to give a mix solution stream (the term “static mixer” refers to mixers which contain fixed mixing devices, e.g. flow pins, past which the materials to be mixed flow and mix with one another as a result of swirling and other disturbed flow; the term “dynamic mixers” refers to mixers which contain active mixing devices, e.g. in the form of rotating mixing blades; in these, the materials to be mixed are mixed with one another by active transport).

It has been found to be useful in practice to aid or exclusively effect mixing in the mixing zone by action of ultrasound. For example, a rod-shaped ultrasonic probe can be inserted into the mixing zone for this purpose.

Of course, the number of inlets of the “T-piece” in the embodiment of the process of the present invention mentioned above by way of example can also be more than two without changing the basic principle of the method.

The mix solution stream produced as described can then be conveyed directly by the shortest route to the atomizer head of a spray dryer (e.g. a Niro Atomizer model Minor Hi-Tec from Niro, Copenhagen, Denmark) and broken up into fine droplets which are dried by contact with hot gas (e.g. air or nitrogen or mixtures of air and nitrogen or noble gases or carbon oxides). The inlet temperature of the hot gas can in the case of the abovementioned spray dryer be, for example, from 200 to 400° C., preferably from 310 to 330° C., in the process of the present invention. The outlet temperature of the drying gas should, according to the present invention, be from 100 to 200° C., preferably from 105 to 115° C. The atomized mix solution and the hot drying gas can be conveyed in cocurrent or in countercurrent in the spray dryer. The droplet size resulting from atomization is usually from 5 to 1 000 μm, frequently from 10 to 100 μm. The drying time of such droplets is less than one second in conventional spray dryers. In principle, spray drying in the process of the present invention can also be carried out as described in EP-A 603836.

The atomization of the total solution in the process of the present invention can be carried out either by means of nozzles (e.g. by means of centrifugal nozzles), by means of gas pressure atomizers or by means of atomizer disks or atomizer baskets (sometimes also called “rotary nozzles”). Atomizer disks and atomizer baskets are preferred according to the present invention. Although they are more complicated in engineering terms and have a higher energy consumption compared to other nozzles, they are less sensitive to solid particles which may be formed. In such atomizers, the total solution generally runs into the middle of the disk or basket without applied pressure, is broken up and is sprayed as a hollow cone from the smooth edge of the disk or from the perforated rim of the basket.

The part solution streams can, in the process of the present invention, also be fed directly to a dynamic mixer as described in DE-A 10043489, micromixers as described in DE-A 10041823 or mixing nozzles as described in DE-A 19958355 and mixed according to the present invention in these. Mixing nozzles of this type used in the process of the present invention can be either smooth stream nozzles, Levo nozzles, Bosch nozzles or jet dispersers. According to the present invention, preference is given to using mixing nozzles which both combine and mix the part solution streams and atomize the resulting mix stream. The atomized total solution can then be dried in cocurrent or in countercurrent by means of hot gases as in a spray dryer. The advantage of the process of the present invention is based on the preparation of stable part solutions which are combined and mixed only when flowing continuously, as a result of which a mix solution stream which can be spray dried with a narrow residence time distribution without time delay is produced directly and in a minimum time.

In general, the process of the present invention from the time at which combination of the two or more part solution streams to form a total solution stream is commenced until dispersion (atomization) of the mix solution stream is complete normally takes less than two minutes, preferably less than one minute, very particularly preferably less than thirty seconds and advantageously in terms of use less than twenty seconds or less than ten seconds. When micromixers as described in DE-A 10041823 are used, this time can even be less than five seconds and in favorable cases even ≦2 sec. or ≦1 sec.

These times are normally sufficient to ensure that solids formation within the mix solution does not take place before it has been atomized completely.

Before the thermal treatment of the solid obtained by drying of the atomized (dispersed) mix solution stream in the process of the present invention, it can, if desired, firstly be tableted (if desired with addition of from 0.05 to 3% by weight of finely divided graphite) and only then treated thermally. After the thermal treatment, tableting can be reversed by milling or crushing.

The thermal treatment can be carried out as described in DE-A 19835247, EP-A 529853, EP-A 603836, EP-A 608838, EP-A 895809, DE-A 19835247, EP-A 962253, EP-A 1080784, EP-A 1090684, EP-A 1123738, EP-A 1192987, EP-A 1192986, EP-A 1192982, EP-A 1192983 and EP-A 1192988.

The thermal treatment (calcination) at from 200 to 1 200° C., or from 300 to 650° C. or from 400 to 600° C., can in principle be carried out either under an oxidizing atmosphere, a reducing atmosphere or an inert atmosphere. A possible oxidizing atmosphere is, for example, air, air enriched with molecular oxygen or air depleted in molecular oxygen. However, according to the present invention, the thermal treatment is preferably carried out under an inert atmosphere, e.g. under molecular nitrogen and/or noble gas. The thermal treatment is usually carried out under atmospheric pressure (1 atm). Of course, the thermal treatment can also be carried out under reduced pressure or under superatmospheric pressure. The temperature in the thermal treatment usually does not exceed 650° C. However, higher temperatures can be advantageous, particularly when the multimetal oxide composition M comprises the element Cs and/or other alkali metals or alkaline earth metals.

If the thermal treatment is carried out under a gaseous atmosphere, this can be static or may flow. The thermal treatment can take a total time of up to 24 hours or more. Higher temperatures correlate with shorter treatment times and vice versa.

The thermal treatment is preferably firstly carried out under an oxidizing (oxygen-containing) atmosphere (e.g. under air) at from 100 to 400° C. or from 200 to 300° C. (=preliminary decomposition step). The thermal treatment is then advantageously continued under inert gas at from 300 to 650° C., or from 400 to 600° C. or from 450 to 600° C.

The multimetal oxide compositions M which can be obtained as described can be used as such (i.e. as powder or granules) or after shaping to give shaped bodies as catalytically active compositions for the partial gas-phase oxidations and/or ammoxidations of saturated and unsaturated hydrocarbons or lower aldehydes described at the beginning of the present text. Here, the catalyst bed can be a fixed bed, a moving bed or a fluidized bed. Shaping can be carried out, for example, by extrusion or tableting in the case of all-active catalysts or by application to a support body (production of coated catalysts), as described in DE-A 10118814 or PCT/EP/02/04073 or DE-A 10051419.

The support bodies to be used for the multimetal oxide compositions M according to the present invention in the case of coated catalysts are preferably chemically inert, i.e. they do not participate significantly in the partial catalytic gas-phase oxidation or ammoxidation of the hydrocarbon (e.g. propane and/or propene to acrylic acid) or aldehyde which is catalyzed by the multimetal oxide compositions M of the present invention.

According to the present invention, suitable materials for the support bodies are, in particular, aluminum oxide, silicon dioxide, silicates such as clay, kaolin, steatite (preferably steatite from CeramTec (Germany) of the type C-220, or preferably having a low water-soluble alkali content), pumice, aluminum silicate and magnesium silicate, silicon carbide, zirconium dioxide and thorium dioxide.

The surface of the support body can be either smooth or rough. The surface of the support body is advantageously rough, since an increased surface roughness generally results in stronger adhesion of the shell of active composition applied.

The surface roughness R_{z} of the support body is frequently in the range from 5 to 200 μm, often in the range from 20 to 100 μm (determined in accordance with DIN 4768 part 1 using a “Hommel Tester for DIN-ISO surface parameters” from Hommelwerke, Germany).

The support material can be porous or unporous. The support material is advantageously nonporous (total volume of pores based on the volume of the support body ≦1% by volume).

The thickness of the active oxide composition layer present in the coated catalysts according to the invention is usually from 10 to 1 000 μm. However, it can also be from 50 to 700 μm, from 100 to 600 μm or from 150 to 400 μm. The thickness of the coating can also be in the range from 10 to 500 μm, from 100 to 500 μm or from 150 to 300 μm.

In principle, all geometries of the support bodies are possible in the process of the present invention. Their maximum dimension is generally from 1 to 10 mm. However, preference is given to using spheres or cylinders, in particular hollow cylinders, as support bodies. Advantageous diameters for support spheres are from 1.5 to 4 mm. If cylinders are used as support bodies, their length is preferably from 2 to 10 mm and their external diameter is preferably from 4 to 10 mm. In the case of rings, the wall thickness is usually from 1 to 4 mm. Ring-shaped support bodies suitable for the purposes of the present invention can have a length of from 3 to 6 mm, an external diameter of from 4 to 8 mm and a wall thickness of from 1 to 2 mm. However, a support ring geometry of 7 mm×3 mm×4 mm or 5 mm×3 mm×2 mm (external diameter×length×internal diameter) is also possible.

The production of the coated catalysts can be most simply carried out by preforming multimetal oxide compositions M according to the present invention, converting them into a finely divided form and subsequently applying them with the aid of a liquid binder to the surface of the support body. For this purpose, the surface of the support body is most simply moistened with the liquid binder and a layer of the active composition is applied to the moistened surface by bringing the surface into contact with finely divided active multimetal oxide composition M. The coated support body is finally dried. Of course, the procedure can be repeated periodically to achieve an increased coating thickness. In this case, the coated body becomes the new “support body” etc.

It goes without saying that the fineness of the catalytically active multimetal oxide composition M of the formula (I) to be applied to the surface of the support body is matched to the desired coating thickness. For coating thicknesses in the range from 100 to 500 μm, active composition powders in which at least 50% of the total number of powder particles pass a sieve having a mesh opening of from 1 to 20 μm and the proportion by number of particles having a maximum dimension above 50 μm is less than 10% are, for example, suitable. In general, the distribution of the maximum dimensions of the powder particles corresponds to a Gaussian distribution as a result of the method of production. The particle size distribution is frequently as follows:

| D (μm) | 1 | 1.5 | 2 | 3 | 4 | 6 | 8 | 12 | 16 | 24 | 32 | 48 | 64 | 96 | 128 |
|---|---|---|---|---|---|---|---|---|---|---|---|---|---|---|---|
| x | 80.5 | 76.3 | 67.1 | 53.4 | 41.6 | 31.7 | 23 | 13.1 | 10.8 | 7.7 | 4 | 2.1 | 2 | 0 | 0 |
| y | 19.5 | 23.7 | 32.9 | 46.6 | 58.4 | 68.3 | 77 | 86.9 | 89.2 | 92.3 | 96 | 97.9 | 98 | 100 | 100 |

In this table:
D = diameter of the particle,
x = the percentage of particles whose diameter is ≧ D; and
y = the percentage of particles whose diameter is < D.

To carry out the coating process described on an industrial scale, it is advisable to employ, for example, the process principle disclosed in DE-A 2909671 or that disclosed in DE-A 10051419, i.e. the support bodies to be coated are placed in a rotating vessel (e.g. a rotating pan or coating drum) which is preferably inclined (the angle of inclination is generally from ≧0° to ≦90°, usually from ≧30° to <90°; the angle of inclination is the angle of the central axis of the rotating vessel to the horizontal). The rotating vessel conveys the for example spherical or cylindrical support bodies through under two metering devices which follow one another at a particular distance. The first of the two metering devices advantageously corresponds to a nozzle (e.g. an atomizer nozzle operated by means of compressed air) by means of which the support bodies rolling in the rotating vessel are sprayed with the liquid binder and moistened in a controlled fashion. The second metering device is located outside the atomization cone of the liquid binder sprayed in and serves to introduce the finely divided oxidic active composition (e.g. by means of a vibratory chute or a powder screw). The support spheres which have been moistened in a controlled fashion take up the active composition powder introduced, which is consolidated to a coherent shell on the outer surface of the for example cylindrical or spherical support body by means of the rolling motion.

If required, the support body which has received its basic coating in this way once again passes under the spray nozzles during the subsequent rotation, is moistened in a controlled fashion and during the further motion is able to take up a further layer of finely divided oxidic active composition, etc. (intermediate drying is generally not necessary). Finely divided oxidic active composition and liquid binder are generally fed in continuously and simultaneously.

After coating is complete, the liquid binder can be removed, for example by action of hot gases such as N₂ or air. It is notable that the coating process described produces fully satisfactory adhesion both of the successive layers to one another and of the base layer to the surface of the support body.

In the above-described coating method, it is important that moistening of the surface of the support body to be coated is carried out in a controlled fashion. Stated briefly, this means that the support surface is advantageously moistened so that although the surface has adsorbed liquid binder, no liquid phase is visible as such on the support surface. If the surface of the support body is too moist, the finely divided catalytically active oxide composition agglomerates to form separate agglomerates instead of becoming attached to the surface. Detailed information on this subject may be found in DE-A 2909671 and in DE-A 10051419.

The abovementioned subsequent removal of the liquid binder used can be carried out in a controlled way, e.g. by evaporation and/or sublimation. In the simplest case, this can be carried out by action of hot gases having an appropriate temperature (frequently from 50 to 300° C., often 150° C.). However, it is also possible for only predrying to be effected by action of hot gases. Final drying can then be carried out, for example, in a drying oven of any type (e.g. belt dryer) or in the reactor. The temperature employed should not be above the calcination temperature used in the preparation of the oxidic active composition. Of course, drying can also be carried out exclusively in a drying oven.

As binders for the coating process, it is possible to use, regardless of the type and geometry of the support body: water, monohydric alcohols such as ethanol, methanol, propanol and butanol, polyhydric alcohols such as ethylene glycol, 1,4-butanediol, 1,6-hexanediol or glycerol, monobasic or polybasic organic carboxylic acids such as propionic acid, oxalic acid, malonic acid, glutaric acid or maleic acid, amino alcohols such as ethanolamine or diethanolamine and also monofunctional or polyfunctional organic amides such as formamide. Useful binders also include solutions consisting of from 20 to 90% by weight of water and from 10 to 80% by weight of an organic compound having a boiling point or sublimation temperature at atmospheric pressure (1 atm) of >100° C., preferably >150° C., dissolved in water. The organic compound is advantageously selected from the above listing of possible organic binders. The proportion of organic component in the abovementioned aqueous binder solutions is preferably from 10 to 50% by weight, particularly preferably from 20 to 30% by weight. Possible organic components also include monosaccharides and oligosaccharides such as glucose, fructose, sucrose or lactose and also polyethylene oxides and polyacrylates.

Possible geometries (both for all-active catalysts and for coated catalysts) are spheres, solid cylinders and hollow cylinders (rings). The maximum dimension of the abovementioned geometries is generally from 1 to 10 mm. In the case of cylinders, their length is preferably from 2 to 10 mm and their external diameter is preferably from 4 to 10 mm. In the case of rings, the wall thickness is usually from 1 to 4 mm. Suitable ring-shaped all-active catalysts can also have a length of from 3 to 6 mm, an external diameter of from 4 to 8 mm and a wall thickness of from 1 to 2 mm. However, an all-active catalyst ring geometry of 7 mm×3 mm×4 mm or 5 mm×3 mm×2 mm (external diameter×length×internal diameter) is also possible. Of course, all the geometries mentioned in DE-A 10101695 are also possible for the active multimetal oxide compositions M.

The specific surface area of multimetal oxide compositions M according to the invention (and also of the multimetal oxide compositions M′, M″ discussed later in this patent application) is frequently from 1 to 40 m²/g, often from 11 or 12 to 40 m²/g and mostly from 15 or 20 to 40 or 30 m²/g (determined by the BET method, nitrogen).

According to the present invention, the stoichiometric coefficient a of the multimetal oxide compositions M obtainable according to the present invention is, independently of the preferred ranges for the other stoichiometric coefficients of the multimetal oxide compositions M, from 0.05 to 0.6, particularly preferably from 0.1 to 0.6 or up to 0.5.

Independently of the preferred ranges for the other stoichiometric coefficients of the multimetal oxide compositions M, the stoichiometric coefficient b is preferably from >0 or 0.01 to 1, particularly preferably from 0.01 or 0.1 to 0.5 or up to 0.4.

The stoichiometric coefficient c of the multimetal oxide compositions M obtainable according to the present invention is, independently of the preferred ranges for the other stoichiometric coefficients of the multimetal oxide compositions M, advantageously from 0.01 to I and particularly preferably from 0.01 or 0.1 to 0.5 or up to 0.4. A very particularly preferred range for the stoichiometric coefficient c, which can, independently of the preferred ranges for the other stoichiometric coefficients of the multimetal oxide compositions M obtainable according to the present invention, be combined with all other preferred ranges in the present text, is the range from 0.05 to 0.2.

The stoichiometric coefficient d of the multimetal oxide compositions M, obtainable according to the present invention is, independently of the preferred ranges for the other stoichiometric coefficients of the multimetal oxide compositions M, preferably from 0.00005 or 0.0005 to 0.5, particularly preferably from 0.001 to 0.5, frequently from 0.002 to 0.3 and often from 0.005 or 0.01 to 0.1.

Independently of the preferred ranges for the other stoichiometric coefficients of the multimetal oxide compositions M, the coefficient e can be from >0 to 0.5.

Particularly advantageous multimetal oxide compositions M obtainable according to the present invention are ones whose stoichiometric coefficients a, b, c and d are simultaneously within the following ranges:

a=0.05 to 0.6;

b=0.01 to 1 (or 0.01 to 0.5);

c=0.01 to 1 (or 0.01 to 0.5);

d=0.0005 to 0.5 (or 0.001 to 0.3); and

e=≧0 to 0.5.

Very particularly advantageous multimetal oxide compositions M obtainable according to the present invention are ones whose stoichiometric coefficients a, b, c and d are simultaneously in the following ranges:

a=0.1 to 0.6;

b=0.1 to 0.5;

c=0.1 to 0.5;

d=0.001 to 0.5, or 0.002 to 0.3, or 0.005 to 0.1; and

e=≧0 to 0.2.

M¹ is preferably Te.

All that has been said above applies especially when at least 50 mol % of the total amount of M² is Nb and/or Ta and very particularly preferably when 75 mol % of the total amount of M², or 100 mol % of the total amount of M², is Nb.

It also applies, independently of the meaning of M², especially when M³ is at least one element from the group consisting of Fe, Ni, Co, Pd, Ag, Au, Pb and Ga or at least one element from the group consisting of Ni, Co, Fe and Pd.

All that has been said above also applies especially when at least 50 mol % of the total amount of M², or at least 75 mol % or 100 mol % of M², is Nb and M³ is at least one element from the group consisting of Ni, Co, Fe, Pd, Ag, Au, Pb and Ga.

All that has been said above also applies especially when at least 50 mol % or at least 75 mol % or 100 mol % of the total amount of M² is Nb and M³ is at least one element from the group consisting of Ni, Co, Fe and Pd.

All statements made in respect of the stoichiometric coefficients very particularly preferably apply when M¹=Te, M²=Nb and M³=at least one element from the group consisting of Ni, Co, Fe and Pd.

Advantageous multimetal oxide compositions M are those (in particular all those mentioned above) in which e=0. If e>0, M⁴ is preferably Cs. M² is then preferably Bi.

Further stoichiometries which are suitable for the purposes of the present invention are those in the present text which are disclosed in the cited prior art for the multimetal oxide compositions of the stoichiometry (I).

Preference is also given, according to the present invention, to multimetal oxide compositions M whose X-ray diffraction pattern displays reflections h, i and k whose maxima are at diffraction angles (2θ) of 22±0.5° (h), 27.3±0.50 (i) and 28.2+0.50 (k), where

the reflection h is the most intense in the X-ray diffraction pattern and has a width at half height of not more than 0.50°,

the intensity Pᵢ of the reflection i and the intensity Pₖ of the reflection k obey the relationship 0.65<R<0.85 where R is the intensity ratio defined by the equation

R═Pᵢ/(Pᵢ+Pₖ)

and

the widths at half height of the reflection i and the reflection k are each ≦1°.

All figures given in the present text with regard to an X-ray diffraction pattern are based on an X-ray diffraction pattern obtained using Cu-K_{α} radiation (Siemens diffractometer Theta-Theta D-5000, tube voltage: 40 kV, tube current: 40 mA, aperture V20 (variable), collimator V20 (variable), secondary monochromator aperture (0.1 mm), detector aperture (0.6 mm), measurement interval (2θ): 0.02°, measurement time per step: 2.4 s, detector: scintillation counter; the definition of the intensity of a reflection in the X-ray diffraction pattern in this text is the definition given in DE-A 19835247, DE-A 10122027, or the definition given in DE-A 10051419 and DE-A 10046672; the same applies to the definition of the width at half height.

According to the present invention, preference is given to R obeying the relationship 0.67≦R≦0.75; R is very particularly preferably from 0.69 to 0.75 or from 0.71 to 0.74 or R=0.72.

Apart from the reflections h, i and k, the X-ray diffraction pattern of multimetal oxide compositions M preferred according to the present invention generally displays further reflections whose maxima are located at the following diffraction angles (2θ):

9.0±0.4° (I),

6.7±0.4° (O) and

7.9±0.4° (p).

It is also advantageous for the X-ray diffraction pattern to additionally display a reflection whose maximum is at a diffraction angle (2θ)=45.2±0.40 (q).

The X-ray diffraction pattern of advantageous multimetal oxide compositions M frequently also contains the reflections 29.2±0.4° (m) and 35.4±0.4° (n) (positions of the maxima).

If the reflection h is assigned an intensity of 100, it is advantageous for the reflections i, l, m, n, o, p, q to have, on the same intensity scale, the following intensities:

i: from 5 to 95, frequently from 5 to 80, sometimes from 10 to 60;

l: from 1 to 30;

m: from 1 to 40;

o: from 1 to 30;

p: from 1 to 30 and

q: from 5 to 60.

If the X-ray diffraction pattern of the multimetal oxide compositions M obtainable according to the present invention additionally contain any of the above-mentioned additional reflections, the width at half height of these is generally <10. Multimetal oxide compositions M obtainable according to the present invention whose X-ray diffraction pattern does not display any reflection having a maximum at 2θ=50.0±0.30° in addition to the abovementioned features (individually or together) are particularly advantageous.

The definition of the intensity of a reflection in the X-ray diffraction pattern in the present text is the definition given in DE-A 19835247 and that in DE-A 10051419 and DE-A 10046672.

If the multimetal oxide compositions M obtainable according to the present invention do not directly conform to the abovementioned advantageous requirement profile, this can generally be attained by washing the multimetal oxide compositions M obtainable according to the present invention with suitable liquids, e.g. as described in DE-A 10254279. Possible liquids for this purpose are, for example, organic acids or their aqueous solutions (e.g. oxalic acid, formic acid, acetic acid, citric acid and tartaric acid) and also inorganic acids and their aqueous solutions (e.g. nitric acid or telluric acid) or else alcohols or hydrogen peroxide and their aqueous solutions. Of course, it is also possible to use mixtures of the abovementioned washing liquids for the purposes of washing. Furthermore, JP-A 7-232071 also discloses a suitable washing process.

Washing leaves multimetal oxide compositions M′ whose stoichiometry generally likewise corresponds to the formula (I) and can be used as catalytically active compositions in the same way as the multimetal oxide compositions M. In general, the multimetal oxide compositions M′ have an advantageous X-ray diffraction pattern of the type described.

Advantageous multimetal oxide compositions (which can be used like multimetal oxide compositions M or multimetal oxide compositions M′) also include multimetal oxide compositions M″ which can be produced from multimetal oxide compositions M obtainable according to the present invention or multimetal oxide compositions M′ which are obtainable therefrom and whose stoichiometric coefficient d is 0 or <0.5 by, for example, impregnating them with solutions (e.g. aqueous solutions) of elements M³ (e.g. by spraying), subsequently drying them (possibly at temperatures of ≦100° C.) and subsequently treating them thermally like the precursor compositions of the multimetal oxide compositions M (preferably in a stream of inert gas; prior decomposition in air is preferably omitted). The stoichiometry of the resulting compositions M″ is advantageously chosen so as to correspond to the formula I for the multimetal oxide compositions M. The use of aqueous carbonate, hydrogencarbonate, nitrate and/or halide solutions of elements M³ and/or the use of aqueous solutions in which the elements M³ are present as complexes with organic compounds (e.g. acetates or acetylacetonates) are/is particularly advantageous in this preparative variant. However, doping of multimetal oxide compositions M or M′ in which d=0 or <0.5 can also be carried out as described in EP-A 1266688 (gas phase deposition).

Of course, the multimetal oxide compositions M obtainable according to the present invention can also be diluted with finely divided, e.g. colloidal, materials such as silicon dioxide, titanium dioxide, aluminum oxide, zirconium oxide and niobium oxide which act essentially only as diluents and then used in diluted form as catalytically active compositions.

The dilution mass ratio can be up to 9 (diluent):1 (active composition), i.e. possible dilution mass ratios are, for example, 6 (diluent):1 (active composition) and 3 (diluent):1 (active composition). The diluents can be incorporated before and/or after calcination, generally even before drying. They can even be incorporated in at least one part solution.

If incorporation is carried out before drying or before calcination, the diluent has to be chosen so that it is essentially retained in the fluid medium or during calcination. This is, for example, generally the case for oxides which have been calcined at appropriately high temperatures.

When the diluent materials are incorporated (e.g. in the form of their sols) in at least one part solution, the term solution for the purposes of the present text also encompasses the total system minus the added diluent material, since this is normally a finely divided insoluble inert solid.

The multimetal oxide compositions M obtainable according to the present invention and also the compositions M′ and M″ are suitable, either as such or in the diluted form just described, as active compositions for heterogeneously catalyzed partial gas-phase oxidations (including oxydehydrogenations) and/or ammoxidations of saturated and/or unsaturated hydrocarbons and of aldehydes.

Such saturated and/or unsaturated hydrocarbons are, in particular, ethane, ethylene, propane, propylene, n-butane, isobutane and isobutene. Target products are, in particular, acrolein, acrylic acid, methacrolein, methacrylic acid, acrylonitrile and methacrylonitrile. However, the multimetal oxide compositions are also suitable for heterogeneously catalyzed partial gas-phase oxidation and/or ammoxidation of compounds such as acrolein and methacrolein.

However, ethylene, propylene and acetic acid can also be target products.

For the purposes of the present text, complete oxidation of the hydrocarbon means that all the carbon present in the hydrocarbon is converted into oxides of carbon (CO, CO₂).

All reactions of the hydrocarbon with molecular oxygen other than this are encompassed by the term partial oxidation in the present text. Additional participation of ammonia in the reaction characterizes partial ammoxidation.

The multimetal oxide compositions M, M′ and M″ obtainable as described in the present text are preferably used as catalytically active compositions for the conversion of propane into acrolein and/or acrylic acid, of propane into acrylic acid and/or acrylonitrile, of propylene into acrolein and/or acrylic acid, of propylene into acrylonitrile, of isobutane into methacrolein and/or methacrylic acid, of isobutane into methacrylic acid and/or methacrylonitrile, of ethane into ethylene, of ethane into acetic acid and of ethylene into acetic acid.

Carrying out such partial oxidations and/or ammoxidations (the reaction can be carried out essentially exclusively as a partial oxidation or exclusively as a partial ammoxidation or as a superposition of the two reactions by selection of the ammonia content of the reaction mixture in a manner known per se; cf., for example, WO 98/22421) is known per se from the multimetal oxide compositions of the stoichiometry I of the prior art and the reaction can be carried out in a fully analogous manner.

If crude propane or crude propylene is used as hydrocarbon, this preferably has the composition described in DE-A 10246119 or DE-A 10118814 or PCT/EP/02/04073.

The procedure described there is likewise preferably employed.

A partial oxidation of propane to acrylic acid to be carried out using multimetal oxide active compositions M (or M′ or M″) as catalysts can be carried out, for example, as described in EP-A 608838, WO 0029106, JP-A 10-36311 and EP-A 1192987.

As source of the molecular oxygen required, it is possible to use, for example, air, oxygen-enriched air or air depleted in oxygen or pure oxygen.

Such a process is also advantageous when the reaction gas starting mixture does not contain any noble gas, in particular no helium, as inert diluent gas. Furthermore, the reaction gas starting mixture can of course comprise inert diluent gases such as N₂, CO and CO₂ in addition to propane and molecular oxygen. According to the present invention, water vapor is advantageous as a constituent of the reaction gas mixture.

In such a case, the reaction gas starting mixture which is to be passed over the multimetal oxide active composition M, M′ or M″ obtainable according to the present invention at reaction temperatures of, for example, from 200 to 550° C. or from 230 to 480° C. or from 300 to 440° C. and pressures of from 1 to 10 bar, or from 2 to 5 bar, can have, for example, the following composition:

from 1 to 15% by volume, preferably from 1 to 7% by volume, of propane,

from 44 to 99% by volume of air and

from 0 to 55% by volume of water vapor.

Preference is given to reaction gas starting mixtures comprising water vapor.

Another possible composition of the reaction gas starting mixture is:

from 70 to 95% by volume of propane,

from 5 to 30% by volume of molecular oxygen and

from 0 to 25% by volume of water vapor.

It is self evident that such a process gives a product gas mixture which does not consist exclusively of acrylic acid. Rather, the product gas mixture further comprises not only unreacted propane but also secondary components such as propene, acrolein, CO₂, CO, H₂O, acetic acid, propionic acid, etc., from which the acrylic acid has to be separated.

This can be carried out in a manner known from the heterogeneously catalyzed gas-phase oxidation of propene to acrylic acid.

In such a separation, the acrylic acid present in the product gas mixture can be separated off by absorption in water or by absorption in a high-boiling inert hydrophobic organic solvent (e.g. a mixture of diphenyl ether and diphyl, which may further comprise additives such as dimethyl phthalate). The resulting mixture of absorption medium and acrylic acid can subsequently be worked up in a manner known per se by rectification, extraction and/or crystallization to give pure acrylic acid. As an alternative, the basic separation of the acrylic acid from the product gas mixture can also be carried out by fractional condensation, as is described, for example, in DE-A 19 924 532.

The aqueous acrylic acid condensate obtained in such a condensation can then be purified further by, for example, fractional crystallization (e.g. suspension crystallization and/or layer crystallization).

The residual gas mixture remaining after the basic separation of the acrylic acid comprises, in particular, unreacted propane which is preferably recirculated fo the gas-phase oxidation. For this purpose, part or all of it can be separated off from the residual gas mixture, e.g. by fractional pressure rectification, and subsequently recirculated to the gas-phase oxidation. However, it is better to bring the residual gas into contact with a hydrophobic organic solvent which is able to absorb the propane preferentially in an extraction apparatus (e.g. by passing the gas through the solvent).

The absorbed propane can be liberated again by subsequent desorption and/or stripping with air and be recirculated to the process of the present invention. Economical total propane conversions can be achieved in this way. Propene formed as secondary component is, as in other separation processes, generally not separated from the propane, or only incompletely separated from the propane, and circulated with the latter. This also applies in the case of other homologous saturated and olefinic hydrocarbons. In particular, it applies quite generally to heterogeneously catalyzed partial oxidations and/or ammoxidations according to the present invention of saturated hydrocarbons.

In these cases, an advantage observed is that the multimetal oxide compositions M, M′ and M″ obtainable according to the present invention are also able to heterogeneously catalyze the partial oxidation and/or ammoxidation of the homologous olefinic hydrocarbon to the same target product.

Thus, acrylic acid can be prepared by heterogeneously catalyzed partial gas-phase oxidation of propene by means of molecular oxygen as described in DE-A 10118814 or PCT/EP/02/04073 or JP-A 7-53448, using the multimetal oxide compositions M, M′ or M″ obtainable according to the present invention as active compositions.

This means that a single reaction zone A is sufficient for carrying out the process. The catalytically active compositions present in this reaction zone are exclusively multimetal oxide catalysts M, M′ and/or M″ obtainable according to the present invention.

This is unusual because the heterogeneously catalyzed gas-phase oxidation of propene to acrylic acid generally occurs in two steps which follow one another in time. Propene is usually oxidized essentially to acrolein in the first step and acrolein formed in the first step is usually oxidized to acrylic acid in the second step.

Conventional processes for the heterogeneously catalyzed gas-phase oxidation of propene to acrylic acid therefore usually employ a specific catalyst type tailored to the respective oxidation step for each of the two abovementioned oxidation steps.

In other words, the conventional processes for the heterogeneously catalyzed gas-phase oxidation of propene to acrylic acid employ two reaction zones, in contrast to the process of the present invention.

It is of course possible for only one or more than one multimetal oxide catalyst M, M′ and/or M″ obtainable according to the present invention to be present in the single reaction zone A in the process for the partial oxidation of propene. The catalysts used can naturally be diluted with inert material as has been recommended, for example, as support material in the present text.

In the process for the partial oxidation of propene, the temperature can be constant along the single reaction zone A or can alter along the reaction zone A and is controlled by means of a heat transfer medium. In the case of a changing temperature, it can increase or decrease.

If the process of the present invention for the partial oxidation of propene is carried out as a fixed-bed oxidation, it is advantageously carried out in a shell-and-tube reactor whose tubes are charged with the catalyst. A liquid heat transfer medium, generally a salt bath, is normally passed around the catalyst tubes.

A plurality of temperature zones along the reaction zone A can then be realized in a simple manner by more than one salt bath being passed around the catalyst tubes in sections along the catalyst tubes.

Viewed over the reactor, the reaction gas mixture is passed through the catalyst tubes either in cocurrent or in countercurrent to the salt bath. The salt bath itself can have a purely parallel flow relative to the catalyst tubes. However, this can of course also be superimposed on a transverse flow. Overall, the salt bath can also have a meandering flow around the catalyst tubes, which may be in cocurrent or in countercurrent relative to the reaction gas mixture when viewed over the reactor.

In the process for the partial oxidation of propene, the reaction temperature can be from 2000 to 500° C. along the entire reaction zone A. It will usually be from 250 to 450° C. The temperature will preferably be from 330 to 420° C., particularly preferably from 350 to 400° C.

The working pressure in the process for the partial oxidation of propene can be either 1 bar, less than 1 bar or more than 1 bar. Typical working pressures according to the present invention are from 1.5 to 10 bar, frequently from 1.5 to 5 bar.

The propene to be used in the process for the partial oxidation of propene does not have to meet any particularly high purity requirements.

As propene for such a process, it is possible to use, as already said and as applies quite generally to all single-or two-stage processes for the heterogeneously catalyzed gas-phase oxidation of propene to acrolein and/or acrylic acid, propene having, for example, one of the following two specifications (also known as crude propene) without any problems at all:

a) Polymer Grade Propylene:

| | |
|---|---|
| ≧99.6% by weight | propene |
| ≦0.4% by weight | propane |
| ≦300 ppm by weight | ethane and/or methane |
| ≦5 ppm by weight | C₄-hydrocarbons |
| ≦1 ppm by weight | acetylene |
| ≦7 ppm by weight | ethylene |
| ≦5 ppm by weight | water |
| ≦2 ppm by weight | O₂, |
| ≦2 ppm by weight | sulfur-containing compounds |
| | (calculated as sulfur) |
| ≦1 ppm by weight | chlorine-containing compounds |
| | (calculated as chlorine) |
| ≦5 ppm by weight | CO₂, |
| ≦5 ppm by weight | CO, |
| ≦10 ppm by weight | cyclopropane |
| ≦5 ppm by weight | propadiene and/or propyne |
| ≦10 ppm by weight | C_{≧5}-hydrocarbons and |
| ≦10 ppm by weight | compounds containing carbonyl |
| | groups (calculated as Ni(CO)₄) |

b) Chemical Grade Propylene:

| | |
|---|---|
| ≧94% by weight | propene |
| ≦6% by weight | propane |
| ≦0.2% by weight | methane and/or ethane |
| ≦5 ppm by weight | ethylene |
| ≦1 ppm by weight | acetylene |
| ≦20 ppm by weight | propadiene and/or propyne |
| ≦100 ppm by weight | cyclopropane |
| ≦50 ppm by weight | butene |
| ≦50 ppm by weight | butadiene |
| ≦200 ppm by weight | C₄-hydrocarbons |
| ≦10 ppm by weight | C_{≧5}-hydrocarbons |
| ≦2 ppm by weight | sulfur-containing compounds |
| | (calculated as sulfur) |
| ≦0.1 ppm by weight | sulfides (calculated as H₂S), |
| ≦1 ppm by weight | chlorine-containing compounds |
| | (calculated as chlorine) |
| ≦0.1 ppm by weight | chlorides (calculated as Cl^{θ}) and |
| ≦30 ppm by weight | water |

Of course, all the abovementioned possible accompanying components in the propene can each be present in from two to ten times the stated individual amount in the crude propene without adversely affecting the usability of the crude propene for the process or for known processes for the single- or two-stage heterogeneously catalyzed gas-phase oxidation of propene to acrolein and/or acrylic acid quite generally.

This applies particularly when the accompanying components are, like the saturated hydrocarbons, the water vapor, the carbon oxides or the molecular oxygen, compounds which are in any case present in relatively large amounts as inert diluent gases or as reactants in the abovementioned process. The crude propene is usually admixed as such with circulating gas, air and/or molecular oxygen and/or diluted air and/or inert gas for use in the process for the heterogeneously catalyzed gas-phase oxidation of propene to acrolein and/or acrylic acid.

As propene source for the process of the present invention, it is also possible to use propene which is formed as by-product in a process different from the process of the present invention and contains, for example, up to 40% of its weight of propane. This propene can be additionally accompanied by other accompanying components which do not interfere significantly in the process of the present invention.

As oxygen source for the process for the partial oxidation of propene, it is possible to use either pure oxygen or air or air which has been enriched with or depleted in oxygen.

Apart from molecular oxygen and propene, a reaction gas starting mixture to be used for the process for the partial oxidation of propene usually further comprises at least one diluent gas. Possible diluent gases are nitrogen, carbon oxides, noble gases and lower hydrocarbons such as methane, ethane and propane (higher hydrocarbons, e.g. C₄-hydrocarbons, should be avoided). Water vapor is frequently also used as diluent gas. Mixtures of gases selected from among those mentioned above are frequently employed as diluent gas for the process for the partial oxidation of propene.

The heterogeneously catalyzed partial oxidation of propene is advantageously carried out in the presence of propane.

The reaction gas starting mixture for the propene oxidation process typically has the following composition (molar ratios):

propene: oxygen: H₂O: other diluent gases=1: (0.1-10): (0-70): (0:20).

The abovementioned ratio is preferably 1: (1-5): (1-40): (0-10).

If propane is used as diluent gas, part of it can, as described, also advantageously be oxidized to acrylic acid.

According to the present invention, the reaction gas starting mixture advantageously comprises molecular nitrogen, CO, CO₂, water vapor and propane as diluent gas.

The molar ratio of propane: propene can be from 0 to 15, frequently from 0 to 10, often from 0 to 5, advantageously from 0.01 to 3, in the propene oxidation process.

The space velocity of propene over the catalyst charge in the process for the partial oxidation of propene can be, for example, from 40 to 250 standard l/l·h. The space velocity of reaction gas starting mixture over the catalyst is frequently in the range from 500 to 15 000 standard 1′-h, frequently in the range from 600 to 10 000 standard l/l·h, often from 700 to 5 000 standard l/l·h.

It is self evident that the process for the partial oxidation of propene to acrylic acid gives a product gas mixture which does not consist exclusively of acrylic acid. Rather, the product gas mixture further comprises unreacted propene and secondary components such as propane, acrolein, CO₂, CO, H₂O, acetic acid, propionic acid, etc., from which the acrylic acid has to be separated.

This can be carried out as is generally known from the two-stage (carried out in two reaction zones) heterogeneously catalyzed gas-phase oxidation of propene to acrylic acid.

In such a separation, the acrylic acid present in the product gas mixture can be separated off by absorption in water or by absorption in a high-boiling inert hydrophobic organic solvent (e.g. a mixture of diphenyl ether and diphyl, which may further comprise additives such as dimethyl phthalate). The resulting mixture of absorption medium and acrylic acid can subsequently be worked up in a manner known per se by rectification, extraction and/or crystallization to give pure acrylic acid. As an alternative, the basic separation of the acrylic acid from the product gas mixture can also be carried out by fractional condensation, as is described, for example, in DE-A 19 924 532.

The aqueous acrylic acid condensate obtained in such a condensation can then be purified further by, for example, fractional crystallization (e.g. suspension crystallization and/or layer crystallization).

The residual gas mixture remaining after the basic separation of the acrylic acid comprises, in particular, unreacted propene (and possibly propane). This can be separated off from the residual gas mixture, e.g. by fractional pressure rectification, and subsequently be recirculated to the gas-phase oxidation according to the present invention. However, it is better to bring the residual gas into contact with a hydrophobic organic solvent which is able to absorb the propene (and possibly propane) preferentially in an extraction apparatus (e.g. by passing the gas through the solvent).

The absorbed propene (and possibly propane) can be liberated again by subsequent desorption and/or stripping with air and be recirculated to the process of the invention. Economical total propene conversions can be achieved in this way. If propene is partially oxidized in the presence of propane, propene and propane are preferably separated off and recirculated together.

The multimetal oxides M, M′ and/or M″ obtainable according to the present invention can be used in a completely analogous manner as catalysts for the partial oxidation of isobutane and/or isobutene to methacrylic acid.

They can be used for the ammoxidation of propane and/or propene as described in, for example, EP-A 529853, DE-A 2351151, JP-A 6-166668 and JP-A 7-232071.

They can be used for the ammoxidation of n-butane and/or n-butene as described in JP-A6-211767.

They can be used for the oxydehydrogenation of ethane to ethylene or the further reaction to give acetic acid as described in U.S. Pat. No. 4,250,346 or EP-B 261264.

They can be used for the partial oxidation of acrolein to acrylic acid as described in DE-A 10261186.

The multimetal oxide compositions M, M′ and/or M″ obtainable according to the present invention can also be integrated into other multimetal oxide compositions (e.g. their finely divided powders can be mixed, if appropriate pressed and calcined, or be mixed as slurries (preferably aqueous), dried and calcined (e.g. as described in EP-A 529853 for multimetal oxide compositions of the stoichiometry I where d=0)). Once again, calcination is preferably carried out under inert gas.

The resulting multimetal oxide compositions (hereinafter referred to as total compositions) preferably comprise ≧50% by weight, particularly preferably ≧75% by weight and very particularly preferably ≧90% by weight or ≧95% by weight, of multimetal oxide compositions M, M′ and/or M″ obtainable according to the present invention and are likewise suitable for the partial oxidations and/or ammoxidations discussed in the present text.

The total compositions also preferably display no reflections having maxima at 2θ=50.0±3.0° in the X-ray diffraction pattern.

If the total composition displays a reflection having a maximum at 20=50.0±3.0°, it is advantageous for the proportion by weight of the multimetal oxide compositions M, M′ and/or M″ obtainable according to the present invention to be >80% by weight or >90% by weight or >95% by weight. Such total compositions can be obtained, for example, by washing not being carried out quantitatively in the process of the present invention for preparing the multimetal oxide compositions M′.

The total compositions are advantageously shaped to give geometric bodies as described for the multimetal oxide compositions M, M′ and/or M″ obtainable according to the present invention.

The advantages of the multimetal oxide compositions M, M′ and/or M″ obtainable according to the present invention are based on their comparatively homogeneous structure which generally results in improved activity and/or selectivity when they are used as active compositions for the partial oxidations or ammoxidations mentioned in the present text.

For the purposes of the heterogeneously catalyzed partial gas-phase oxidation of propane to acrylic acid, the multimetal oxide compositions M, M′ and/or M″ and multimetal oxide compositions or catalysts in which these are present are preferably employed as described in DE-A 10122027.

### EXAMPLES AND COMPARATIVE EXAMPLES

A) Production of a Coated Catalyst S1 which Bears a Multimetal Oxide Composition M Obtained According to the Present Invention

To prepare a part solution A, 4 000 ml of water were firstly heated to 80° C. in a glass vessel. While maintaining the temperature at 80° C. and while stirring, 706.2 g of ammonium heptamolybdate from H. C. Starck, Goslar (Germany) having an MoO₃ content of 81.53% by weight (=4 mol of Mo) were dissolved therein. Likewise at 80° C., 141.0 g of ammonium metavanadate from H. C. Starck, Goslar (Germany) having a V₂O₅ content of 77.4% by weight (=1.2 mol of V) were stirred into the resulting clear solution and dissolved therein. Once again at 80° C., 211.28 g of Te(OH)₆ from Fluka Chemie GmbH, Buchs (Switzerland) having a Te(OH)₆ content of ≧99% (=0.92 mol of Te) were stirred into the resulting clear solution and dissolved therein. The resulting reddish solution was cooled to 25° C. and water having a temperature of 25° C. was added while stirring to give a clear, transparent part solution A having a total volume of 4 500 ml.

To prepare a part solution B, 221.28 g of niobium ammonium oxalate from H. C. Starck, Goslar (Germany) having an Nb content of 20.1% by weight (0.48 mol of Nb) were dissolved in 1 000 ml of water which had been heated to 80° C. The resulting clear, transparent solution was cooled to 25° C. and water which likewise had a temperature of 25° C. was added to give a clear, transparent part solution B having a total volume of 1 500 ml.

The two stable aqueous solutions A and B were subsequently pumped continuously by means of two ProMinent laboratory metering pumps, model gamma g/4a, via two separate plastic hoses into the two inlet pieces of a Y-shaped plastic T-piece. The three tubular parts of the T-piece (2 inlet pieces and 1 outlet piece) each had an internal diameter of 5 mm and a length of 38 mm. The solution A was conveyed at a flow rate of 1 500 ml/h and the solution B was conveyed at a flow rate of 500 ml/h. In the interior of the T-piece, the two solution streams A and B were combined to give a total solution stream of 2 000 ml/h which flowed into the outlet piece of the T-piece. A static mixer model SMXS from Sulzer Chemtech, Obermorlen-Ziegenberg (Germany) was located in the latter. The diameter of the static mixer was 4.8 mm, and the length of the mixer rod was 35 mm. The end of the outlet piece of the T-piece was connected directly to the atomizer head of a spray dryer (Niro Atomizer, model Minor Hi-Tec from Niro, Copenhagen (Denmark)) which atomized the mix solution stream fed in (droplet size about 30 μm). Within the atomizer head, which was located in the center of the hot air distributor affixed at the top of the spray dryer, the mix solution stream flowed through a 15 cm long connecting line having an internal diameter of 6 mm directly onto an atomizer disk (channel disk) rotating at 30 000 revolutions per minute. The resulting spray mist was dried by a stream of hot air (cocurrent, inlet temperature 320° C., outlet temperature 105° C.). The entire 6 000 ml of total solution stream were able to be spray dried over a period of 3 hours.

From the total solution flow rate of 2 000 ml/h, the internal diameter of the T-piece outlet and the length of the static mixing section of 35 mm, it is possible to calculate a time t¹ of about 1.2 seconds within which the combined part solution streams A and B are converted into an essentially homogeneous mix solution stream. If the transport of the mix solution stream from the outlet of the static mixer through the 15 cm long connecting line in the atomizer head having an internal diameter of 6 mm to the point of atomization is additionally taken into account, a time t² of less than nine seconds from the combination of the solution streams A and B to atomization of their mix solution stream is calculated. If a drying time of less than one second is included, the time t³ from the combination of the solutions to the dry powder is less than ten seconds. Corresponding to the stoichiometry of solution A and solution B derived from the quantities weighed out and the chosen part solution flows (3:1), the elements Mo, V, Nb and Te are present in the resulting spray-dried powder in a molar stoichiometry of Mo₁V_{0.3}Nb_{0.12}Te_{0.23} (when the outlet piece of the T-piece was not connected directly to the atomizer head of the spray dryer but instead a 15 cm long, transparent plastic hose having an internal diameter of 6 mm was connected to the end of the T-piece outlet and the mix solution stream was transported through this into a collection vessel located below, visual monitoring indicated that the mix solution stream contained no precipitate over the entire length of the plastic hose and when it arrived in the collection vessel and was all clear and transparent; a filtration experiment on the mix solution flowing from the plastic hose confirmed the freedom from solids).

150 g of the resulting spray-dried powder were heated from room temperature (25° C.) to 275° C. at a heating rate of 5° C./min in air (10 standard l/h) in a rotary sphere furnace as shown in FIG. 1 of DE-A 10118814. Immediately afterwards, the powder was heated from 275° C. to 650° C. at a heating rate of 2° C./min in a stream of molecular nitrogen (10 standard l/h) and this temperature was held for 6 hours while maintaining the flow of nitrogen. The powder was subsequently allowed to cool naturally to 25° C. while maintaining the flow of nitrogen. A black calcined multimetal oxide active composition M was obtained.

The calcined material was milled in a Retsch mill (centrifugal mill, model ZM 100, from Retsch, Germany) (particle size ≦0.12 mm). 75 g of the resulting powder were applied to 162 g of spherical support bodies having a diameter of 2.2-3.2 mm (R_{z}=45 μm; support material=steatite C 220 from Ceramtec (Germany), total pore volume of the support ≦1% by volume based on the total volume of the support). For this purpose, the support spheres were placed in a coating drum having an internal volume of 2 l (angle of inclination of the central axis of the drum to the horizontal=30°). The drum was set into rotation at 25 revolutions per minute. A total of 30 ml of a mixture of glycerol and water (weight ratio of glycerol:water=1:3) was sprayed uniformly onto the initially charged support spheres over a period of 60 minutes by means of an atomizer nozzle operated using 300 standard l/h of compressed air. The nozzle was installed so that the spray cone wetted the support bodies conveyed in the drum by means of conveyor projections to the uppermost point of the inclined drum in the upper half of the roll-down section. The active composition powder was introduced into the drum by means of a powder screw, with the point of introduction of the powder being located within the roll-down section or below the spray cone. As a result of the periodic repetition of wetting and application of powder, the initially coated support body itself became the support body in the subsequent period.

After coating had been completed, coated support bodies were dried at 120° C. for 16 hours in a convection drying oven (from Binder (Germany), internal volume=53 l). The glycerol was removed by a subsequent heat treatment at 150° C. for 2 hours in air.

A coated catalyst S1 having an active composition content of 32% by weight was obtained.

B) Production of a Coated Catalyst S2 Bearing a Multimetal Oxide Composition M Obtained According to the Present Invention

The procedure employed in A) was repeated, but the part solution stream A was 3 000 ml/h (instead of 1 500 ml/h) and the part solution stream B was 1 000 ml/h (instead of 500 ml/h). In addition, the inlet temperature of the spray dryer was set to 400° C. instead of 320° C.

In this case, t¹ is calculated as about 0.6 seconds, t² as 4.5 seconds and t³ as 5.5 seconds. The 6 000 ml of total solution stream were spray dried over a period of 1.5 hours. The stoichiometry of the spray-dried powder was likewise Mo₁V_{0.3}Nb_{0.12}Te_{0.23}. The resulting coated catalyst was the coated catalyst S2:

C) Production of a Coated Catalyst S3 Bearing a Multimetal Oxide Composition M′ Obtainable from a Multimetal Oxide Composition M Obtained According to the Present Invention

100 g of a multimetal oxide composition M obtained as described in A) after calcination were added to 500 g of 20% strength by weight aqueous nitric acid.

The resulting aqueous suspension was stirred at 70° C. under reflux for 7 hours. It was then cooled to 25° C. The solid present in the black suspension was separated from the aqueous phase by filtration, washed free of nitrate by means of water and subsequently dried overnight at 120° C. in a convection drying oven. The dried material was subsequently milled in a Retsch mill in a manner analogous to procedure A) (particle size ≦00.12 mm) and the resulting powder was processed further as described in A) to give a coated catalyst S3.

D) Production of a Coated Catalyst S4 Bearing a Multimetal Oxide Composition M′ Obtainable from a Multimetal Oxide Composition M Obtained According to the Present Invention

100 g of a multimetal oxide composition M obtained as in B) after calcination were treated with aqueous nitric acid as described in C) and the solid which remained was processed further as described in C) to give a coated catalyst S4.

E) Production of a Coated Catalyst CS5 Bearing a Multimetal Oxide Composition Obtained by a Method which is not According to the Present Invention

As described in A), 4 500 ml of part solution A and 1 500 ml of part solution B, each at a temperature of 25° C., were prepared. The 1 500 ml of part solution B were then stirred into part solution A within about 3 seconds. This gave 6 000 ml of a reddish, clear, transparent mix solution C having a temperature of 25° C. Immediately afterwards, the continuously stirred mix solution C was, while maintaining the temperature of 25° C., conveyed continuously by means of a ProMinent laboratory metering pump, model gamma g/4a, via a plastic hose at a volume flow of 2 000 ml/h to the atomizer head of the spray dryer employed in A) and, as described in A), atomized in this and dried in a stream of hot air (inlet temperature 320° C., outlet temperature 105° C.).

While the mix solution C was still clear and transparent during the first two minutes, pronounced turbidity was observed after only 2.5 minutes. After 5 minutes, a significant amount of a reddish solid had precipitated and after 1 hour a completely opaque reddish aqueous suspension had been formed. Thus, while a homogeneous spray-dried powder was still obtained from a clear, transparent solution at the commencement of spray drying, an inhomogeneous spray-dried powder containing an increasing proportion of the precipitated reddish solid, whose composition deviated significantly from the quantities weighed out and dissolved initially to give the mix solution C, was obtained in the further course of spray drying (while the stoichiometry based on the quantities weighed out was Mo₁V_{0.3}Nb_{0.12}Te_{0.23}, the elemental composition of the solid separated off by filtration after 1 hour was Mo₁V_{0.3}Nb_{0.6}Te_{0.4}; the X-ray diffraction pattern of the reddish solid displayed three very broad peaks in the 2θ range from 5 to 65°, with the peak having the maximum amplitude being at about 28°).

The spray-dried powder obtained after all the solution/suspension had been spray dried was homogeneously mixed and treated thermally (calcined) and processed further as described in A) to give a coated catalyst CS5.

F) Production of a Coated Catalyst CS6 Bearing a Multimetal Oxide Composition Obtained from a Multimetal Oxide Composition Obtained by a Method which is not According to the Present Invention

The multimetal oxide obtained after calcination in E) was treated with aqueous nitric acid as described in C) and the solid which remained was processed further as described in C) to give a coated catalyst CS6.

G) Testing of the Coated Catalysts S1 to CS6

A reaction tube made of V2A steel (length: 140 cm, external diameter: 60 mm, internal diameter: 8.5 cm) was in each case charged with the respective coated catalyst. The length of the catalyst bed was set to 52 cm (accommodated in the middle of the reaction tube). A preliminary bed having a length of 30 cm and made up of steatite (C220 from CeramTec, diameter: 2.2-3.2 mm) served to preheat the reaction gas mixture. The reaction tube downstream of the catalyst zone was subsequently filled with the same steatite balls. The reaction tube was heated from the outside over its entire length by means of electric heating mats. The mat temperature was set to 340° C. The reaction was carried out at a pressure of 2 bar absolute, a residence time (based on the catalyst bed) of 2.4 s using a feed (reaction gas starting mixture) having the molar composition propane:air:water=1:15:14. The selectivity S (mol %) of acrylic acid formation in a single pass through the reaction tube was determined by gas-chromatographic analysis of the product gas stream. The results listed in the following table were obtained for the coated catalysts used.

The propane conversion C (mol %) and the selectivity of acrylic acid formation were each arbitrarily set to 100 for the coated catalyst S1.

TABLE

| Coated catalyst | C [mol %] | S [mol %] |
|---|---|---|
| S1 | 100 | 100 |
| S2 | 107.7 | 104.5 |
| S3 | 207.7 | 168.2 |
| S4 | 223 | 170.5 |
| CS5 | 92.3 | 93.2 |
| CS6 | 200 | 163.6 |

H) Repetition According to the Present Invention of the “Preparation of Complex Metal Oxide (1)” from JP-A 11-306228

29.21 liters of water were heated to 80° C. While maintaining the temperature of 80° C., 7.09 kg of ammonium paramolybdate tetrahydrate ((=ammonium heptamolybdate) from H. C. Starck, Goslar (Germany) having an MoO₃ content of 81.53% by weight), 1.41 kg of ammonium metavanadate (from H. C. Starck, Goslar (Germany) having a V₂O₅ content of 77.4% by weight) and 2.12 kg of telluric acid (from Fluka Chemie GmbH, Buchs (Switzerland) having a Te(OH)₆ content of >99%) were then successively dissolved therein. 5 kg of silica sol having an SiO₂ content of 20% by weight (produced from 2.5 kg of Ludox AS-40 colloidal silica 40 wt. % suspension in water, DuPont product, Aldrich Chem. Comp. Inc., Milwaukee, USA, and 2.5 kg of water) were finally added to the resulting solution, and the solution was cooled to 50° C. so as to give a part solution A.

2.16 kg of ammonium niobium oxalate (from H. C. Starck, Goslar (Germany) having an Nb content of 20.1% by weight) were dissolved in 8.66 liters of water which had been heated to 80° C. The solution was then cooled to 50° C. so as to give a part solution B.

The part solutions A and B, each at a temperature of 50° C., were combined, mixed and spray dried as described in A) (part solution stream A: 1 570 ml/h, part solution stream B: 430 ml/h).

Examination as in A) showed that the mix solution stream contain no precipitate up to the time when it was spray dried (the sol used was likewise clear and transparent).

We claim:

1. A process for preparing a multimetal oxide composition M of the stoichiometry I
Mo₁VₐM¹_{b}M²_{c}M³_{d}M⁴ₑOₙ (1),
where
M¹=at least one element from the group consisting of Te and Sb;
M²=at least one element from the group consisting of Nb, Ti, W, Ta, Bi, Zr and Re;
M³=at least one element from the group consisting of Pb, Ni, Co, Fe, Pd, Ag, Pt, Cu, Au, Ga, Zn, Sn, In, Ce, Ir, Sm, Sc, Y, Pr, Nd and Tb;
M⁴=at least one element from the group consisting of Li, Na, K, Rb, Cs, Ca, Sr, Ba;
a=0.01 to 1,
b=>0 to 1,
c=>0 to 1,
d=>0 to 0.5,
e=>0 to 1 and
n=a number which is determined by the valence and abundance of elements other than oxygen in (I),
in which a mix solution is produced continuously in a solvent from the required starting compounds of the elemental constituents of the multimetal oxide composition M, the mix solution is fed continuously into a drying apparatus for removing the solvent and the solid obtained is treated thermally at elevated temperature, with the thermal treatment comprising a calcination at from 200 to 1 200° C., wherein at least two physically separate part solutions each containing partial amounts of the required starting compounds of the elemental constituents of the multimetal oxide composition M in dissolved form are firstly prepared, at least two part solution streams are produced from the two or more part solutions, the two or more part solution streams are combined to form a total solution stream, the total solution stream is passed through a mixing zone in which a mix solution stream comprising the total amount of the required starting compounds in dissolved form is formed, the mix solution stream is either broken up into fine droplets in the mixing zone or the mix solution stream is discharged from the mixing zone and then broken up into fine droplets, the fine droplets of mix solution are dried by contact with hot gas and the solid obtained is treated thermally at elevated temperature, with the thermal treatment comprising a calcination at from 200 to 1 200° C.

2. A process as claimed in claim 1, wherein the solids content of the mix solution stream, expressed as total content of the metals present, is from 1 to 30% by weight.

3. A process as claimed in claim 1, wherein the solids content of the mix solution stream, expressed as total content of the metals present, is from 5 to 20% by weight.

4. A process as claimed in any of claims 1 to 3, wherein the solvent used is an aqueous solvent.

5. A process as claimed in any of claims 1 to 4, wherein the temperature of the two or more part solution streams is from 15 to 40° C.

6. A process as claimed in any of claims 1 to 4, wherein the temperature of the two or more part solution streams is from 20 to 30° C.

7. A process as claimed in any of claims 1 to 6, wherein the number of part solutions is from 2 to 5.

8. A process as claimed in any of claims 1 to 7, wherein the process from the time at which combination of the two or more part solution streams to form a total solution stream is commenced until the breaking-up of the mix solution stream is complete takes less than two minutes.

9. A process as claimed in any of claims 1 to 7, wherein the process from the time at which combination of the two or more part solution streams to form a total solution stream is commenced until the breaking-up of the mix solution stream is complete takes less than thirty seconds.

10. A process as claimed in any of claims 1 to 7, wherein the process from the time at which combination of the two or more part solution streams to form a total solution stream is commenced until the breaking-up of the mix solution stream is complete takes less than twenty seconds.

11. A process as claimed in any of claims 1 to 10, wherein a=0.05 to 0.6.

12. A process as claimed in any of claims 1 to 11, wherein b=0.01 to 1.

13. A process as claimed in any of claims 1 to 12, wherein c=0.01 to 1.

14. A process as claimed in any of claims 1 to 13, wherein d=0.0005 to 0.5.

15. A process as claimed in any of claims 1 to 14, wherein
a=0.1 to 0.6;
b=0.1 to 0.5;
c=0.1 to 0.5;
d=0.001 to 0.5 and
e=≧0 to 0.5.

16. A process as claimed in any of claims 1 to 15, wherein at least 50 mol % of M² is Nb and/or Ta.

17. A process as claimed in any of claims 1 to 16, wherein M³ is at least one element from the group consisting of Ni, Co, Fe and Pd.

18. A process as claimed in any of claims 1 to 17, wherein M¹=Te, M²=Nb and M³ is at least one element from the group consisting of Ni, Fe, Co and Pd.

19. A process as claimed in any of claims 1 to 18, wherein at least one part solution comprises at least one added finely divided diluent material from the group consisting of silicon dioxide, titanium dioxide, aluminum oxide, zirconium oxide and niobium oxide.

20. A process for the heterogeneously catalyzed partial gas-phase oxidation and/or ammoxidation of saturated and/or unsaturated hydrocarbons, wherein a multimetal oxide composition M obtained by a process as claimed in any of claims 1 to 19 is used as active composition.

21. A multimetal oxide composition M obtainable by a process as claimed in any of claims 1 to 19.
